(19) United States
(12) Patent Application Publication
(10) Pub. No.: US 2026/0248656 A1
(43) Pub. Date: Aug. 27, 2026

(54) SEALING STRIP

(71) Applicant: ConvaTec Limited, Flintshire (GB)

(72) Inventors: Natalie Brown, Newton-Le-Willows (GB); Liam Davies, Sheffield (GB); Beth Gaskell, Irby (GB); Annchalee Eade, Northwich (GB); Donna Kesteven, Upton (GB); Lucy Ballamy, Llangollen (GB)

(21) Appl. No.: 19/651,029

(22) Filed: Apr. 17, 2026

Related U.S. Application Data
(63) Continuation of application No. PCT/GB2024/052660, filed on Oct. 17, 2024.
(60) Provisional application No. 63/591,614, filed on Oct. 19, 2023.

(30) Foreign Application Priority Data
Dec. 7, 2023 (GB) 2318726.3

(51) Int. Cl. A61F 13/05; A61F 13/02; A61F 13/0246

(52) U.S. Cl. CPC A61F 13/05; A61F 13/0253; A61F 13/0266

(57) ABSTRACT

The invention provides a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner.

### TECHNICAL FIELD OF THE INVENTION

The present invention relates to a sealing strip for a negative pressure wound dressing and a negative pressure wound therapy kit comprising at least one sealing strip.

### BACKGROUND TO THE INVENTION

Negative pressure wound therapy (NPWT) is a wound dressing system wherein a sub-atmospheric pressure is applied intermittently or continuously to a wound site. NPWT can be used on acute or chronic wounds and can be advantageous for a patient because it can lead to quicker healing and reduced likelihood of infection.

Typically negative pressure wound therapy dressings are secured to a patient's skin by an adhesive surface on the dressing. Additional sealing strips can then be applied to the edges of the wound dressing to ensure that a tight seal is formed such that the sub-atmospheric pressure can be successfully applied to the wound without significant air leakage. Frequent handling of part or all of the NPWT dressing could introduce bacteria into the wound thereby leading to infection; therefore, to avoid having to frequently replace the sealing strip or the entire NPWT dressing it would be advantageous to provide a sealing strip with an increased wear time.

Improved wear time can be achieved through high adhesion, but high adhesion could also result in the sealing strip being painful to remove when it does need to be removed or replaced, for example to examine the wound or change the dressing to a clean dressing. It would therefore be advantageous to provide a sealing strip with improved adhesion leading to increased wear time which, when required, doesn't require excess force to remove or cause pain to the patient.

Sealing strips require a high moisture vapour transfer rate (MVTR) so that moisture can be removed from the skin and does not become trapped between the sealing strip and the skin. Trapped moisture can negatively impact the adhesion of the sealing strip and cause maceration of the peri-wound skin. Improved MVTR can be achieved with thinner sealing strips however thinner sealing strips can be more difficult to apply. If the sealing strip is difficult to apply it may crease or fold on application and therefore would not have as long a wear time and an increased likelihood of the strips detaching from the wound dressing and skin during use. It would therefore be advantageous to provide a sealing strip which has both an improved MVTR and improved handling and therefore improved wear time.

The ease of application of the sealing strip is essential to the smooth application of the sealing strip which in turn leads to long wear time. In use, a clinician will apply the sealing strip to a patient wearing gloves and may encounter problems such as the sealing strip adhering to itself such that it is not usable, or difficulty removing release layer(s) or protective liner(s) which are required to maintain a sterile environment on the surface of the sealing strip. It would therefore be advantageous to provide a sealing strip which is easy to apply to a patient such that it can lead to an improved wear time on the patient.

It is an aim of embodiments of the invention to overcome one or more problems of the prior art, whether expressly disclosed herein or not.

### SUMMARY OF THE INVENTION

According to a first aspect of the invention there is provided a sealing strip for use in negative pressure wound therapy, comprising:
a carrier layer comprising a front face and a back face;
a protective liner on the back face of the carrier layer;
an adhesive layer on the front face of the carrier layer;
a first release liner on a first portion of the adhesive layer;
a second release liner on a second portion of the adhesive layer, and which extends to one lateral edge of the adhesive layer;
wherein each of the first and second release liners comprises a portion detachably adhered to the adhesive layer and at least one handling tab portion which is not adhered to the adhesive layer;
and wherein the surface area of the handling tab portion or the surface area of the handling tab portions combined of the first release liner is at least 45% of the total surface area of the first release liner.

The sealing strip may comprise opposite longitudinal edges and opposite lateral edges. The sealing strip may be substantially rectangular. The adhesive layer, carrier layer, protective liner and first and second release liners may comprise opposite longitudinal edges and opposite lateral edges. The adhesive layer, carrier layer, protective liner and first and second release layers may be substantially rectangular. The adhesive layer, carrier layer, protective liner and first and second release layers may be substantially rectangular comprising opposite longitudinal edges and opposite lateral edges wherein the longitudinal edges are longer than the lateral edges.

In some embodiments, the adhesive layer comprises a back face, connected to the front face of the carrier layer, and a front face, connected to the first and second release liners.

In some embodiments the first release liner extends to one lateral edge of the adhesive layer and the second release liner extends to an opposite lateral edge of the adhesive layer. The portion of the first release liner which is detachably adhered to the adhesive layer may cover no more than 45% of the total surface area of the adhesive layer. The portion of the first release liner which is detachably adhered to the adhesive layer may cover no more than 44%, 43%, 42%, 41%, 40% 38%, 36%, 34%, 32%, 30%, 28% or no more than 27% of the total surface area of the adhesive layer. The ratio of the size of the portion of the first release liner which is detachably adhered to the adhesive layer to the size of the portion of the first release liner which is the handling tab may be between 1:1 and 1:4. The ratio of the size of the portion of the first release liner which is detachably adhered to the adhesive layer to the size of the portion of the first release liner which is the handling tab may be between 1:1 and 1:3.5, 1:1 and 1:3, 1:1 and 1:2.5, 1:1 and 1:2 or between 1:1 and 1:1.6. This may be advantageous because larger handling tabs may allow a clinician to more easily remove the release liners, especially when wearing gloves. This may be advantageous because smaller portions which are detachably adhered to the adhesive layer, which are exposed on removal of the release liner, may allow a clinician to apply the adhesive layer more easily to the patient's skin in use without the adhesive layer adhering to itself, folding or wrinkling thereby leading to a smoother application.

In some embodiments the sealing strip comprises:
a first release liner on a central portion of the adhesive layer;
at least one second release liner on a second portion of the adhesive layer, and which extends to a lateral edge of the adhesive layer;
at least one third release liner on a third portion of the adhesive layer, and which extends to the opposite lateral edge of the adhesive layer as the second release liner; and
wherein the first, second and third release liners each comprise a portion detachably adhered to the adhesive layer and at least one handling tab portion which is not adhered to the adhesive layer.

The portion of the first release liner which is detachably adhered to the adhesive layer may cover no more than 30% of the total surface area of the adhesive layer. The portion of the first release liner which is detachably adhered to the adhesive layer may cover no more than 29%, 28% or 27% of the total surface area of the adhesive layer. This may be advantageous because in use the portion of the adhesive layer which is exposed by removal of the first release liner is small and therefore there is reduced risk of the adhesive layer folding, wrinkling, or adhering to itself. This may be advantageous in use because it allows the sealing strip to be more easily applied by a clinician.

In some embodiments the portions of each of the release liners which are adhered to the adhesive layer are non-overlapping on the adhesive layer.

In some embodiments the surface area of the handling tab portion or the surface area of the handling tab portions combined of the first release liner is at least 50% of the total surface area of the first release liner. The handling tab portion or the surface area of the handling tab portions combined of the first release liner may be at least 55%, 60%, 65%, 70% or at least 73% of the total surface area of the first release liner. This embodiment may be advantageous because the larger handling tabs may allow a clinician to remove the first release liner more easily, especially when they may be wearing gloves.

The release liners may comprise paper release liners. The release liners may comprise a polymer film. The polymer may be selected from polypropylene, polyethylene, polyurethane or polyester or any combination thereof. The polymer may be selected from the group consisting of: polypropylene, polyethylene, polyurethane and polyester or any combination thereof. The release liners may be polymer coated paper release liners. The release liners may comprise siliconized paper release liners, also known as silicone coated paper release liners.

The release liners may have a weight of between 50 gsm and 80 gsm. The release liners may have a weight of between 55 gsm and 80 gsm, or between 55 gsm and 75 gsm, or between 55 gsm and 70 gsm, or between 55 gsm and 65 gsm or a weight of between 60 gsm and 65 gsm.

In some embodiments the surface area of the handling tab portion of the second release liner is at least 18% of the total surface area of the second release liner. In some embodiments the surface area of the handling tab portion of the third release liner is at least 18% of the total surface area of the third release liner. In some embodiments the surface area of the handling tab portion of the third release liner is at least 19%, 20% or at least 21% of the total surface area of the third release liner. In some embodiments the surface area of the handling tab portion of the second release liner is at least 18% of the total surface area of the second release liner and the surface area of the handling tab portion of the third release liner is at least 18% of the total surface area of the third release liner. In some embodiments the surface area of the handling tab portion of the second release liner is at least 18% of the total surface area of the second release liner and the surface area of the handling tab portion of the third release liner is at least 20% of the total surface area of the third release liner. This embodiment may be advantageous because the larger handling tabs of the second and third release liners may allow a clinician to remove the second and third release liners more easily, especially when they may be wearing gloves. Easier removal of the second and third release liners may prevent the clinician from touching the adhesive layer when applying the adhesive layer to a patient's skin, thereby preventing wrinkling, folding or stretching of the adhesive layer.

In some embodiments the portion of the second release liner which is detachably adhered to the adhesive layer may cover between 20% and 45% of the total surface area of the adhesive layer. In some embodiments the portion of the second release liner which is detachably adhered to the adhesive layer may cover between 25% and 40%, 25% and 38%, 30% and 38%, 32% and 38%, or between 32% and 36% of the total surface area of the adhesive layer.

In some embodiments the portion of the third release liner which is detachably adhered to the adhesive layer may cover between 20% and 45% of the total surface area of the adhesive layer. In some embodiments the portion of the third release liner which is detachably adhered to the adhesive layer may cover between 25% and 40%, 25% and 38%, 30% and 38%, 32% and 38%, or between 32% and 36% of the total surface area of the adhesive layer.

In some embodiments the protective liner comprises at least one portion which extends beyond a first lateral edge of the carrier layer. In some embodiments the protective liner comprises at least one portion which extends beyond a second lateral edge of the carrier layer. In some embodiments the protective liner comprises at least one portion which extends beyond a first lateral edge of the carrier layer and at least one portion which extends beyond a second lateral edge of the carrier layer. The protective liner may comprise a portion in contact with the back face of the carrier layer and at least one handling tab portion, which in some embodiments may be at one or both ends of the protective liner. At least one handling tab portion may comprise at least part of at least one portion which extends beyond a first lateral edge of the carrier layer and/or a second lateral edge of the carrier layer. The protective liner may comprise a portion which adheres to substantially the entire back face of the carrier layer, and one or two handling tab portions which do not adhere to the carrier layer and extend past one or both lateral edges of the carrier layer.

The total surface area of the handling tab portion(s) of the protective liner may be at least 5% of the total surface area of the protective liner. The total surface area of the handling tab portion(s) of the protective liner may be at least 6%, 7%, 8%, 9% or at least 10% of the total surface area of the protective liner.

The handling tab portion of the protective liner may comprise at least one perforated line or area set in from a lateral edge of the protective liner. The perforated line or area may extend across the entire width of the protective liner. The perforated line or area may be located towards the lateral edge of the protective liner. The perforated region may be located inward or inboard of the handling tab portion of the protective liner. Thus, the protective liner may comprise, outermost (lateral) to innermost, a handling portion, a perforated region and an adhesive portion. The perforated region may also comprise an adhesive portion or may be part of the adhesive portion. The perforated line or perforated area may be advantageous because it allows the user to easily remove the protective liner from the back face of the carrier liner without detaching the sealing strip from the patient's skin in use, thereby ensuring the wear time of the sealing strip on the patient's skin is not reduced.

The protective liner may be a paper protective liner. The protective liner may be a polymer film. The polymer may be selected polypropylene, polyethylene, polyurethane or polyester or any combination thereof. The polymer may be selected from the group consisting of: polypropylene, polyethylene, polyurethane and polyester or any combination thereof. The protective liner may be a polymer coated paper protective liner. The protective liner may be a siliconized paper protective liner also known as a silicone coated protective liner.

The protective liner may have a weight of between 15 gsm and 50 gsm. The protective liner may have a weight of between 15 gsm and 45 gsm, 15 gsm and 40 gsm, 18 gsm and 40 gsm, 18 gsm and 35 gsm, 18 gsm and 30 gsm, or a weight of between 18 gsm and 25 gsm.

In some embodiments the adhesive layer comprises an acrylic adhesive. The acrylic adhesive may be chosen from ethyl acrylate, butyl acrylate and methyl acrylate or any combination thereof. The acrylic adhesive may be made from an acrylate selected from the group consisting of: ethyl acrylate, butyl acrylate and methyl acrylate or any combination thereof.

The acrylic adhesive may comprise no more than 20 wt. % solvent. The acrylic adhesive may comprise no more than 18 wt. %, 15 wt. %, 12 wt. %, 10 wt. %, 8 wt. % or no more than 5 wt. % solvent, or essentially no solvent. In some embodiments the adhesive layer is an essentially solvent free adhesive layer. This embodiment may be advantageous because it is more environmentally friendly due to the low concentration of volatile organic compounds.

The adhesive layer may have a weight of no more than 33 gsm. The adhesive layer may have a weight of no more than 32 gsm, 31 gsm, 30 gsm, 29 gsm, 28 gsm, 27 gsm, 26 gsm, or no more than 25 gsm. The adhesive layer may have a weight of no more than 22 gsm, 20 gsm, 18 gsm, 16 gsm or no more than 15 gsm. This embodiment may be advantageous because the low weight, thin adhesive layer results in an improved moisture vapour transfer rate so that moisture is successfully removed from the skin and does not become trapped between the skin and the adhesive layer which can have a negative impact on the adhesion of the sealing strip.

In some embodiments the carrier layer comprises a polymer. The polymer may be chosen from polylactic acid, polycaprolactone, polyethylene (LDPE, HDPE), polypropylene, polyester, polyamide, ethylene vinyl acetate and polyurethane or any combination thereof. The polymer may be selected from a group consisting of: polylactic acid, polycaprolactone, polyethylene (LDPE, HDPE), polypropylene, polyester, polyamide, ethylene vinyl acetate and polyurethane or any combination thereof. In preferred embodiments the carrier comprises polyurethane.

The carrier layer may have a weight of between 20 gsm and 35 gsm, or between 22 gsm and 30 gsm, or between 24 gsm and 30 gsm, or between 25 gsm and 29 gsm or between 25 gsm and 28 gsm.

The carrier layer may be transparent. The carrier layer may be translucent. A transparent or translucent carrier layer may be advantageous because it may allow the user to accurately place the sealing strip at the desired site.

In some embodiments the adhesive layer has a weight of no more than 33 gsm, the carrier layer has a weight of between 25 gsm and 35 gsm, and the release liner(s) has a weight of between 50 gsm and 80 gsm. In some embodiments the adhesive layer has a weight of no more than 30 gsm, the carrier layer has a weight of between 25 gsm and 35 gsm, and the release liner(s) has a weight of between 50 gsm and 80 gsm. In some embodiments the adhesive layer has a weight of no more than 30 gsm, the carrier layer has a weight of between 25 gsm and 28 gsm, and the release liner(s) has a weight of between 50 gsm and 80 gsm. In some embodiments the adhesive layer has a weight of no more than 30 gsm, the carrier layer has a weight of between 25 gsm and 35 gsm, and the release liner(s) has a weight of between 55 gsm and 70 gsm. In some embodiments the adhesive layer has a weight of no more than 30 gsm, the carrier layer has a weight of between 25 gsm and 28 gsm, and the release liner(s) has a weight of between 55 gsm and 70 gsm. In some embodiments the adhesive layer has a weight of no more than 29 gsm, the carrier layer has a weight of between 22 gsm and 30 gsm and the release liner(s) has a weight of between 50 gsm and 80 gsm. In some embodiments the adhesive layer has a weight of no more than 29 gsm, the carrier layer has a weight of between 25 gsm and 30 gsm and the release liner(s) has a weight of between 55 gsm and 75 gsm. In some embodiments the adhesive layer has a weight of no more than 27 gsm, the carrier layer has a weight of between 25 gsm and 30 gsm and the release liner(s) has a weight of between 55 gsm and 60 gsm. In some embodiments the adhesive layer has a weight of no more than 27 gsm, the carrier layer has a weight of between 24 gsm and 30 gsm and the release liner(s) has a weight of between 50 gsm and 80 gsm. In some embodiments the adhesive layer has a weight of no more than 25 gsm, the carrier layer has a weight of between 20 gsm and 30 gsm and the release liner(s) has a weight of between 50 gsm and 80 gsm. In some embodiments the adhesive layer has a weight of no more than 25 gsm, the carrier layer has a weight of between 20 gsm and 30 gsm and the release liner(s) has a weight of between 55 gsm and 65 gsm. In some embodiments the adhesive layer has a weight of no more than 25 gsm, the carrier layer has a weight of between 25 gsm and 30 gsm and the release liner(s) has a weight of between 50 gsm and 80 gsm. In some embodiments the adhesive layer has a weight of no more than 25 gsm, the carrier layer has a weight of between 25 gsm and 30 gsm and the release liner(s) has a weight of between 55 gsm and 65 gsm. In some embodiments the adhesive layer has a weight of no more than 25 gsm, the carrier layer has a weight of between 25 gsm and 28 gsm and the release liner(s) has a weight of between 50 gsm and 80 gsm. In some embodiments the adhesive layer has a weight of no more than 25 gsm, the carrier layer has a weight of between 25 gsm and 28 gsm and the release liner(s) has a weight of between 55 gsm and 65 gsm. This embodiment is advantageous because the thin adhesive layer and thin carrier layer results in a sealing strip with improved MVTR and the release liners result in a sealing strip which can be applied with reduced folding or wrinkling in use, which in turn results in a longer wear time.

The thickness of the carrier layer and the adhesive layer may be no more than 0.058 mm. The thickness of the carrier layer and the adhesive layer may be no more than 0.057, 0.056, 0.055, 0.054, 0.053, 0.052 or preferably no more than 0.051 mm. The reduced thickness of the carrier layer and the adhesive layer may be advantageous because it may result in a sealing strip with improved MVTR. The reduced thickness of the carrier layer and the adhesive layer in combination with the release liners may result in a sealing strip with improved MVTR and reduced folding or wrinkling in use which in turn results in a longer wear time.

In some embodiments the adhesive layer may cover at least 50% of the front face of the carrier layer. The adhesive layer may cover at least 55%, 60%, 65%, 70%, 75%, 80%, 85%, 90%, 95%, 96%, 97%, 98% or at least 99% of the front face of the carrier layer. In some embodiments the adhesive layer covers the entire front face of the carrier layer.

In preferred embodiments there is provided a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer is an essentially solvent free adhesive layer.

In preferred embodiments there is provided a sealing strip for use in negative face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % solvent.

In preferred embodiments there is provided a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising essentially no solvent.

In preferred embodiments there is provided a sealing strip for use in negative face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the first and the second release liners have a weight of between 50 gsm and 80 gsm.

In preferred embodiments there is provided a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the carrier layer has a weight of between 20 gsm and 35 gsm.

In preferred embodiments there is provided a sealing strip for use in negative face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the carrier layer has a weight of between 20 gsm and 35 gsm.

In preferred embodiments there is provided a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the first and the second release liners have a weight of between 50 gsm and 80 gsm.

In preferred embodiments there is provided a sealing strip for use in negative pressure wound therapy, comprising: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, and wherein the adhesive layer comprises an acrylic adhesive comprising no more than 20 wt. % or essentially no solvent, wherein the carrier layer has a weight of between 20 gsm and 35 gsm and wherein the first and the second release liners have a weight of between 50 gsm and 80 gsm.

According to a second aspect of the invention there is provided a negative pressure wound therapy kit comprising a negative pressure wound therapy dressing and at least one sealing strip according to the first aspect of the invention.

The negative pressure wound therapy kit may comprise at least two sealing strips. The negative pressure wound therapy kit may comprise at least 4 sealing strips. The negative pressure wound therapy kit may comprise at least 6 sealing strips. The sealing strips may be detachably connected to each other at at least one edge of the sealing strip, which may be by way of a perforated line or join.

The negative pressure wound therapy kit may comprise a negative pressure wound therapy dressing, a negative pressure source and at least one sealing strip. The negative pressure source may be a pump.

The negative pressure source may be removably connectable to the wound dressing. The negative pressure source may be removably connectable to the wound dressing by way of a connector. The connector may be a valve. The connector may be a one-way valve.

The negative pressure source may be removably connectable to the wound dressing by way of a conduit or tube. The negative pressure source may be removably connectable to the wound dressing by way of a connector and a conduit/tube.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer is an essentially solvent free adhesive layer.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % solvent.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising essentially no solvent.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the first and the second release liners have a weight of between 50 gsm and 80 gsm.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the carrier layer has a weight of between 20 gsm and 35 gsm.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the carrier layer has a weight of between 20 gsm and 35 gsm.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the first and the second release liners have a weight of between 50 gsm and 80 gsm.

In preferred embodiments there is provided a negative pressure wound therapy kit comprising a negative pressure wound therapy dressing and at least one sealing strip wherein the sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent, wherein the carrier layer has a weight of between 20 gsm and 35 gsm and wherein the first and the second release liners have a weight of between 50 gsm and 80 gsm.

According to a third aspect of the invention there is provided a negative pressure wound therapy apparatus comprising a negative pressure wound therapy dressing, a negative pressure source and at least one sealing strip according to the first aspect of the invention, and wherein the negative pressure source is removably connectable to the wound dressing.

The negative pressure wound therapy apparatus may comprise at least two sealing strips. The negative pressure wound therapy apparatus may comprise at least four sealing strips.

The negative pressure source may be removably connected to the wound dressing by way of a connector. The negative pressure source may be removably connected to the wound dressing by way of a conduit or tube.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer is an essentially solvent free adhesive layer, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % solvent, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising essentially no solvent, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the first and the second release liners have a weight of between 50 gsm and 80 gsm, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the carrier layer has a weight of between 20 gsm and 35 gsm, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the carrier layer has a weight of between 20 gsm and 35 gsm, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent and wherein the first and the second release liners have a weight of between 50 gsm and 80 gsm, and wherein the negative pressure source is removably connectable to the wound dressing.

In preferred embodiments there is provided a negative pressure wound therapy apparatus comprising a negative pressure wound therapy dressing, a negative pressure source and at least one sealing strip wherein the or each sealing strip comprises: a carrier layer comprising a front face and a back face; a protective liner on the back face of the carrier layer; an adhesive layer on the front face of the carrier layer; a first release liner on a first portion of the adhesive layer; a second release liner on a second portion of the adhesive layer, and which extends to one lateral edge of the adhesive layer; wherein each of the first and second release liners comprises a portion detachably adhered to the adhesive layer and at least one handling tab portion which is not adhered to the adhesive layer; and wherein the surface area of the handling tab portion or the surface area of the handling tab portions combined of the first release liner is at least 45% of the total surface area of the first release liner, wherein the adhesive layer comprises an acrylic adhesive comprising no more than 20 wt. % or essentially no solvent, wherein the carrier layer has a weight of between 20 gsm and 35 gsm and wherein the first and the second release liners have a weight of between 50 gsm and 80 gsm, and wherein the negative pressure source is removably connectable to the wound dressing.

### DETAILED DESCRIPTION OF THE INVENTION

In order that the invention may be more clearly understood, embodiments thereof will now be described, by way of example only, with reference to the accompanying drawings, of which:

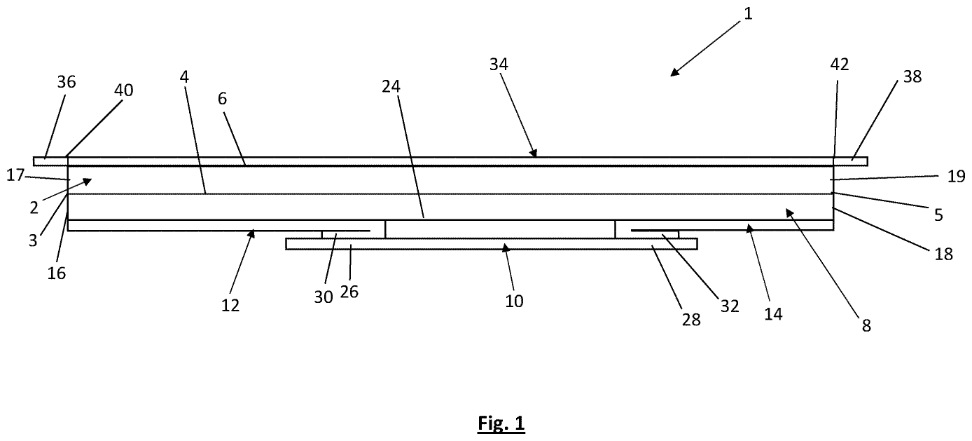

FIG. 1 illustrates a side cross sectional view of a first embodiment of a sealing strip (1) of the first aspect of the invention.

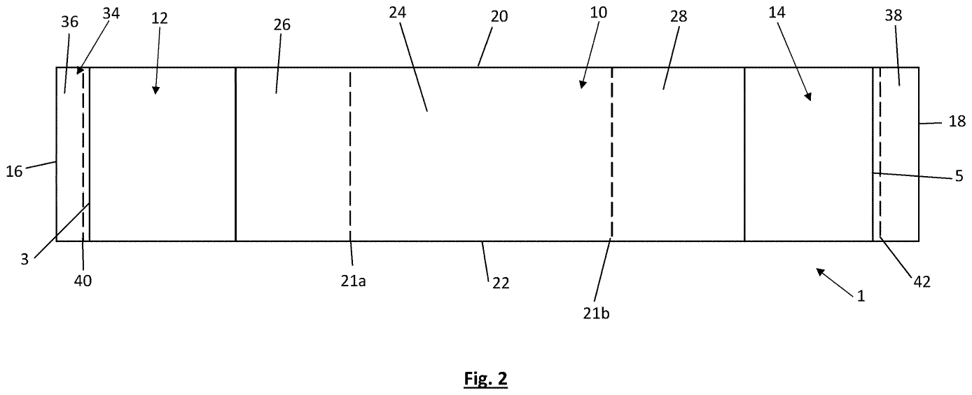

FIG. 2 illustrates a bottom-up view of the first embodiment of the sealing strip (1) of FIG. 1.

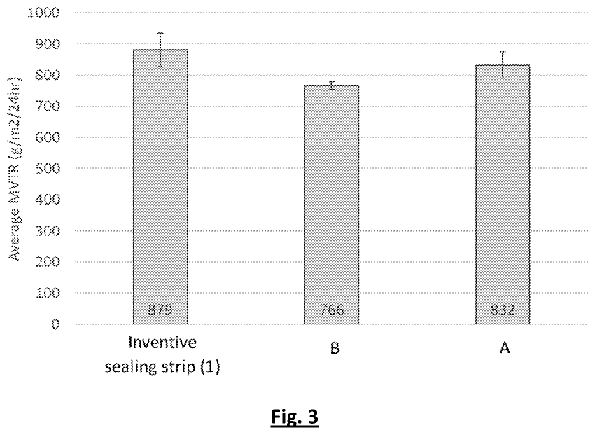

FIG. 3 is a graph of the average moisture vapour transfer rate of the first embodiment of the sealing strip of the invention (1) and competitor sealing strips A and B.

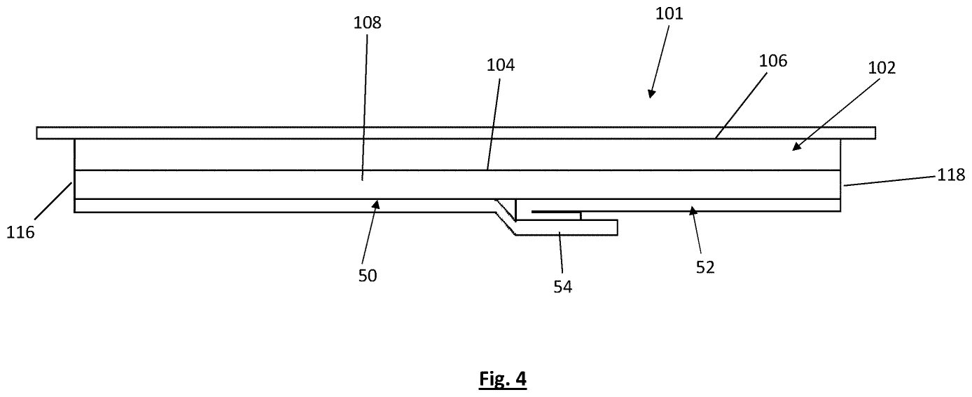

FIG. 4 illustrates a side cross-sectional view of a second embodiment of a sealing strip (101) of the first aspect of the invention.

A first embodiment of a sealing strip (1) of the first aspect of the invention is illustrated in FIG. 1 and FIG. 2. The sealing strip (1) is a rectangular shape comprising two opposing lateral short edges (3, 5) which are connected by two opposing long edges (20, 22). The sealing strip (1) comprises a central portion (24) illustrated by dashed lines (21a, 21b) in FIG. 2. The sealing strip (1) comprises a carrier layer (2) comprising a front face (4) and an opposite back face (6); a protective liner (34) on the back face (6) of the carrier layer (2); an adhesive layer (8) on the front face (4) of the carrier layer (2); a first release liner (10) on the central portion (24) of the adhesive layer (8); a second release liner (12) on a second portion of the adhesive layer (8) and which extends from the central portion (24) to a first lateral edge (16) of the adhesive layer (8) and a third release liner (14) on a third portion of the adhesive layer (8), and which extends from the central portion (24) to a second lateral edge (18) of the adhesive layer (8). The adhesive layer (8) covers essentially the entire front face (4) of the carrier layer (2).

The first release liner (10) is detachably connected to a central portion (24) of the adhesive layer (8). A first portion of the first release liner (10) comprises a first handling tab (26) and a second portion of the first release liner (10) comprises a second handling tab (28). The total surface area of the handling tab portions (26 and 28) of the first release liner (10) as a percentage of the total surface area of the first release liner (10) is 58% of the total surface area of the first release liner (10). The surface area of the central portion (24) of the adhesive layer (8) which is revealed once the first release liner (10) is removed, is 27% of the total surface area of the adhesive layer (8).

The second release liner (12) extends from the edge of the central portion (21a) of the adhesive layer (8) to the first lateral edge (16) of the adhesive layer (8). A portion of the second release liner (12) located towards the edge of the central portion (21b) of the adhesive layer (8) comprises a handling tab (30). The surface area of the handling tab (30) is 18% of the total surface area of the second release liner (12).

The third release liner (14) extends from a second edge of the central portion (21b) of the adhesive layer (8) to the second lateral edge (18) of the adhesive layer (8). A portion of the third release liner (14) located towards edge of the central portion (21b) of the adhesive layer (8) comprises a handling tab (32). The surface area of the handling tab (32) is 21% of the total surface area of the third release liner (14).

The protective liner (34) comprises a portion detachably connected to the back face (6) of the carrier liner (2) and a first handling tab (36) which extends out beyond a first lateral edge (17) of the carrier layer (2) and a second handling tab (38) which extends beyond a second lateral edge (19) of the carrier layer (2). The surface area of the first handling tab (36) of the protective liner (34) is approximately 3% of the total surface area of the protective liner (34). The first handling tab (36) of the protective liner (34) comprises a perforated line (40) which extends along the width of the protective liner (34) from the first longitudinal edge (20) to the second longitudinal edge (22). The second handling tab (38) of the protective liner (34) comprises a perforated line (42) which extends along the width of the protective liner (34) from the first longitudinal edge (20) to the second longitudinal edge (22). The surface area of the second handling tab (38) of the protective liner (34) is approximately 3% of the total surface area of the protective liner (34). The total surface area of the handling tab portions (40 and 42) of the protective liner (34) is therefore approximately 6% of the total surface area of the protective liner (34).

The adhesive layer (8) comprises an acrylic adhesive and the acrylic adhesive is an essentially solvent free acrylic adhesive. The adhesive layer (8) has a weight of approximately 25 gsm. The carrier layer (2) is a polyurethane material. The carrier layer (2) is flexible and translucent. The carrier layer (2) has a weight of approximately 28 gsm. The release liners (10, 12, 14) are siliconized paper wherein the weight of the siliconized paper is 63 gsm. The protective liner (34) is a polyurethane material. The protective liner (34) is flexible and translucent. The protective liner (34) has a weight of approximately 20 gsm. In other embodiments the adhesive layer (8) has a weight of no more than 30 gsm, 29 gsm, 27 gsm or no more than 26 gsm, the carrier layer (2) has a weight of between 20 gsm and 35 gsm, the release liners (10, 12, 14) have a weight of between 50 gsm and 80 gsm, and the protective liner (34) has a weight of between 15 gsm and 30 gsm.

A first embodiment of the negative pressure wound dressing kit according to the second aspect of the invention is provided wherein the negative pressure wound dressing kit comprises: a negative pressure wound dressing comprising a connector wherein the connector is detachably connected to a negative pressure source; and at least two, preferably six sealing strips of the first embodiment of the sealing strip (1) of the invention.

A first embodiment of the negative pressure wound therapy apparatus according to the third aspect of the invention is provided wherein the negative pressure wound therapy apparatus comprises a negative pressure wound therapy dressing, a negative pressure source and at least one sealing strip (1) of the first embodiment of the sealing strip of the invention, and wherein the negative pressure source is removably connectable to the wound dressing.

In use the negative pressure wound dressing is applied to a patient's wound and pressed firmly onto the skin. A sealing strip (1) is then applied to each edge of the wound dressing to ensure that a tight seal is formed to allow for a negative pressure to be applied without air leakage around the wound dressing.

In use, the sealing strip (1) is applied according to the following method:
1) The first release liner (10) is removed from the sealing strip (1) by way of the first handling tab (26) to reveal the central portion (24) of the adhesive layer (8);
2) The central portion (24) of the adhesive layer (8) is applied over the edge of the wound dressing and the patient's skin and pressed firmly;
3) The second release liner (12) is removed by way of the handling tab (30) such that the adhesive layer (8) is exposed, the exposed portion of the adhesive layer (8) is then pressed firmly onto the edge of the wound dressing and the skin;
4) The third release liner (14) is removed by way of the handling tab (32) such that the adhesive layer (8) is exposed, the adhesive layer (8) is then pressed firmly onto the skin; and
5) The protective liner (34) is removed from the back face (6) of the sealing strip (1).
   Alternatively in use, the sealing strip (1) is cut in half and one half is applied according to the following method;
1) The first release liner (10) is removed from the sealing strip (1) by way of the first handling tab (26) to reveal a first exposed portion of the adhesive layer (8);
2) The first exposed portion of the adhesive layer (8) is applied over the edge of the wound dressing and pressed firmly;
3) The second release liner (12) is removed by way of the handling tab (30) and a second exposed portion of the adhesive layer (8) is then pressed firmly onto the skin; and
4) The protective liner (34) is removed from the back face (6) of the sealing strip (1).

The other half of the sealing strip (1) may then be applied in the same manner.

The sealing strip (1) is advantageous because the sealing strip (1) is breathable due to the polyurethane carrier layer (2), the thin acrylic adhesive layer (8) results in strong adhesive properties and a good moisture vapour transfer rate and, the larger total surface area of the handling tabs of each of the release liners and the smaller central adhesive portion (24), ensure that the sealing strip (1) can be smoothly applied to the skin. Together, these features result in a sealing strip (1) which results in longer wear time and is easier to apply. The acrylic adhesive layer (8) also results in less painful removal of the sealing strip (1).

The thickness of the adhesive layer (8) and the carrier layer (2) of the inventive sealing strip (1) compared to 3 sealing strips of the prior art (A, B and C) are shown in table 1.

TABLE 1

| | Average thickness (adhesive |
|---|---|
| Sealing Strip | layer and carrier layer), /mm |
| Inventive sealing strip (1) | 0.0507 |
| Known sealing strip A | 0.0590 |
| Known sealing strip B | 0.0683 |
| Known sealing strip C | 0.0653 |

Sealing strips A, B and C comprise a larger central portion of the adhesive layer which is revealed on removal of a first release liner compared to the central portion (24) of the inventive sealing strip (1). The handling tab portions of the first, second and third release liners of sealing strips A, B and C are smaller than the handling tab portions (26, 28, 30, 32) of the inventive sealing strip (1).

Data demonstrating the performance of the sealing strip (1) on healthy patients compared to 3 sealing strips of the prior art (A, B and C) is shown in table 2 which contains data from 24 healthy volunteers. The data shows the mean percentage of lifting over the 7 days, the pain on removal (mean pain score out of 10 wherein 0 is no pain and 10 is the worst pain imaginable), the number of participants who report moderate to excessive force required to remove the sealing strip.

TABLE 2

| | | Pain on | Number of |
|---|---|---|---|
| | Mean | removal | patients reporting |
| | percentage | (mean pain | force required to |
| Sealing Strip | of lifting | score) | remove |
| Inventive sealing strip (1) | 35% | 0.74 | 38% |
| Known sealing strip A | 19% | 2.52 | 75% |
| Known sealing strip B | 72% | 0.27 | 8% |
| Known sealing strip C | 80% | 0.50 | 8% |

Known sealing strip A comprises an acrylic medical adhesive on a clear polyurethane carrier. Known sealing strip B comprises a 40 gsm acrylic medical adhesive on a clear polyurethane carrier. Known sealing strip C comprises a 38 μm acrylic medical adhesive on a clear polyurethane carrier.

The inventive sealing strip (1) showed a low percentage of lifting and a low pain on removal score which is advantageous for users because it results in a sealing strip which does not require frequent changing as does not easily lift from the skin, but when it does require changing it does not hurt the patient. Known sealing strip A showed a lower percentage of lifting after 7 days but a high number of patients experienced higher pain on removal and stated that excess force was required to remove the sealing strip. A lower number of patients experienced higher pain on removal of known sealing strips B and C however a higher percentage of lifting occurred after 7 days suggesting the wear time of B and C would be shorter.

The inventive sealing strip (1) is advantageous because it provides an excellent balance in terms of preventing unwanted lifting and providing low pain and force to remove the strip, since strip A has better lifting but higher pain and strips B and C have lower pain but worse lifting.

FIG. 3 illustrates the average MVTR for the inventive sealing strip (1) compared to known sealing strips A and B. The sealing strip of the invention (1) demonstrates superior MVTR which is advantageous because it ensures that moisture vapour is not trapped between the skin and the sealing strip (1). Trapped moisture vapour would negatively impact the adhesive properties of the sealing strip (1) and therefore high MVTR improves adhesive properties and is correlated to increased wear time. Trapped moisture vapour would also cause maceration of the peri-wound skin which in turn would lead to pain or discomfort for the patient, and also may lead to an increased risk of infection; therefore, high MVTR is advantageous because it reduces the risk of maceration of the peri-wound skin.

The inventive sealing strip (1) is therefore advantageous because it results in a sealing strip which, when applied in use, has reduced wrinkling or reduced over stretching and an increased MVTR, reduced lifting and reduced pain on removal; all of which lead to a sealing strip with a longer wear time and less risk of peri-wound skin maceration due to less trapped moisture and smoother application.

A second embodiment of a sealing strip (101) of the first aspect of the invention is illustrated in FIG. 4. The sealing strip (101) comprises a carrier layer (102) having opposite front (104) and rear surfaces (106); an adhesive layer (108) covering at least a portion of the front surface (104) and wherein the adhesive layer (108) comprises an acrylic adhesive; and two release liners (50, 52) detachably connected to the adhesive layer (108); wherein the adhesive layer (108) has a weight of 25 gsm, and wherein the carrier layer (102) has a weight of 28 gsm. In other embodiments the adhesive layer (108) has a weight of no more than 30 gsm, 29 gsm, 27 gsm or no more than 26 gsm and the carrier layer (102) has a weight of between 20 gsm and 35 gsm.

The second embodiment of the sealing strip (101) is essentially the same as the first embodiment of the sealing strip with the exception that there are two release liners (50, 52) instead of three. The first release liner (50) extends from the centre of the sealing strip (101) to the first short edge (116) of the sealing strip (101). The second release liner (52) extends from the centre of the sealing strip (101) to the first short edge (118) of the sealing strip (101). A portion (54) of the first release liner (50) overlaps with the second release liner (52) and the portion (54) forms a handling tab. The surface area of the handling tab portion (54) of the first release liner (50) is 57% of the surface area of the first release liner (50). The portion of the first release liner (50) which is in contact with the adhesive layer (108) is in contact with 26% of the adhesive layer (108).

The second release liner (52) comprises a handling tab portion (56) wherein the surface area of the handling tab portion (56) is 21% of the total surface area of the second release liner (52). The portion of the second release liner (52) which is in contact with the adhesive layer (108) is in contact with 74% of the adhesive layer (108).

The second embodiment of the sealing strip (101) may be used in essentially the same way as the first embodiment of the sealing strip with the exception that the step comprising the removal of the third release liner is not required due to the absence of a third release liner.

A second embodiment of the negative pressure wound dressing kit according to the second aspect of the invention is provided wherein the negative pressure wound dressing kit comprises: a negative pressure wound dressing comprising a connector wherein the connector is detachably connected to a negative pressure source; and at least two, preferably six sealing strips (101) of the second embodiment of the sealing strip of the invention.

A second embodiment of the negative pressure wound therapy apparatus according to the third aspect of the invention is provided wherein the negative pressure wound therapy apparatus comprises a negative pressure wound therapy dressing, a negative pressure source and at least one sealing strip (101) of the second embodiment of the sealing strip of the invention, and wherein the negative pressure source is removably connectable to the wound dressing.

The above embodiments are described by way of example only. Many variations are possible without departing from the scope of the invention as defined in the appended claims.

1. A sealing strip for use in negative pressure wound therapy, comprising:
a carrier layer comprising a front face and a back face;
a protective liner on the back face of the carrier layer;
an adhesive layer on the front face of the carrier layer;
a first release liner on a first portion of the adhesive layer;
a second release liner on a second portion of the adhesive layer, and which extends to one lateral edge of the adhesive layer;
wherein each of the first and second release liners comprises a portion detachably adhered to the adhesive layer and at least one handling tab portion which is not adhered to the adhesive layer;
and wherein the surface area of the handling tab portion or the surface area of the handling tab portions combined of the first release liner is at least 45% of the total surface area of the first release liner.

2. A sealing strip according to claim 1 wherein the first release liner extends to one lateral edge of the adhesive layer and the second release liner extends to an opposite lateral edge of the adhesive layer.

3. A sealing strip according to claim 1 or claim 2 wherein the portion of the first release liner which is detachably adhered to the adhesive layer covers no more than 45% of the area of the sealing strip, such as no more than 30% of the area of the sealing strip.

4. A sealing strip according to any preceding claim wherein the sealing strip comprises:
a first release liner on a central portion of the adhesive layer;
at least one second release liner on a second portion of the adhesive layer, and which extends to a lateral edge of the adhesive layer;
at least one third release liner on a third portion of the adhesive layer, and which extends to the opposite lateral edge of the adhesive layer as the second release liner; and
wherein the first, second and third release liners each comprise a portion detachably adhered to the adhesive layer and at least one handling tab portion which is not adhered to the adhesive layer.

5. A sealing strip according to claim 4 wherein the portion of the first release liner which is detachably adhered to the adhesive layer covers no more than 30% of the area of the sealing strip, such as no more than 27% of the area of the sealing strip.

6. A sealing strip according to any preceding claim wherein each of the release liners is non-overlapping on the adhesive layer.

7. A sealing strip according to any preceding claim wherein the surface area of the handling tab portion of the first release liner is at least 50% of the total surface area of the first release liner.

8. A sealing strip according to any preceding claim wherein each of the release liners are paper release liners.

9. A sealing strip according to any preceding claim wherein the surface area of the handling tab portion of each second release liner is at least 18% of the total surface area of the second release liner and/or the surface area of the handling tab portion of each third release liner is at least 18% of the total surface area of the third release liner.

10. A sealing strip according to any preceding claim wherein the protective liner comprises a portion in contact with the back face of the carrier layer and at least one handling tab portion at one or both ends.

11. A sealing strip according to claim 10 wherein at least one handling tab portion of the protective liner comprises at least part of at least one portion which extends beyond the first lateral edge of the carrier layer and/or the second lateral edge of the carrier layer.

12. A sealing strip according to claim 10 or claim 11 wherein the handling tab portion of the protective liner comprises at least one perforated line or area.

13. A sealing strip according to any of claim 10 to claim 12 wherein the total surface area of the handling tab portion(s) of the protective liner is at least 18% of the total surface area of the protective liner.

14. A sealing strip according to any preceding claim wherein the adhesive layer comprises a solvent free acrylic adhesive.

15. A sealing strip according to any preceding claim wherein the weight of the adhesive layer is no more than 30 gsm, or no more than 25 gsm.

16. A sealing strip according to any preceding claim wherein the weight of the carrier layer is between 20 and 35 gsm.

17. A negative pressure wound therapy kit comprising a negative pressure wound therapy dressing and at least one sealing strip according to any one of claims 1 to 16.

18. A negative pressure wound therapy apparatus comprising a wound dressing, a negative pressure source and at least one sealing strip wherein the sealing strip is according to any one of claims 1 to 16 and wherein the negative pressure source is removably connectable to the wound dressing.

19. A negative pressure wound therapy apparatus according to claim 18 wherein the apparatus comprises at least 2 sealing strips, at least 4 sealing strips or at least 6 sealing strips.

20. A negative pressure wound therapy apparatus according to claim 18 or claim 19 wherein the negative pressure source is removably connected to the wound dressing by way of a connector, a conduit or a tube.
